(19) United States
(12) Patent Application Publication
(10) Pub. No.: US 2023/0218263 A1
(43) Pub. Date: Jul. 13, 2023

(54) Retrograde and Independently Articulatable Nested Catheter Systems for Combined Imaging and Therapy Delivery or Other Uses

(71) Applicant: Project Moray, Inc., Belmont, CA (US)

(72) Inventors: Mark D. Barrish, Belmont, CA (US); Keith Phillip Laby, Oakland, CA (US)

(73) Assignee: Project Moray, Inc., Belmont, CA (US)

(21) Appl. No.: 18/125,084

(22) Filed: Mar. 22, 2023

Related U.S. Application Data
(63) Continuation of application No. PCT/US2021/052378, filed on Sep. 28, 2021.
(60) Provisional application No. 63/084,198, filed on Sep. 28, 2020.

(51) Int. Cl. A61B 8/08

(52) U.S. Cl. CPC A61B 8/0841; A61B 8/0883; A61B 34/30

(57) ABSTRACT

Devices, systems, and methods are provided for image-guided interventional procedures and other uses. Nested articulated catheter shaft systems may have an imaging catheter with an ultrasound transducer supported by a fluid-driven articulated sheath portion. Drive fluid can be transmitted distally along an asymmetric sheath via eccentric passages to an articulated portion of the imaging catheter distal of a port. An articulated shaft supporting a therapeutic tool can be advanced within a working lumen of the imaging sheath to the port so that the tool is within a field of view of the transducer. The fluid transmission channels may take much less cross-sectional area of the sheath than a mechanical pull-wire system, allowing the nested sheath/shaft system to provide safer access to a chamber of the heart and to facilitate precise independent control over 3D ultrasound imaging and image-guided structural heart therapies or the like.

### CROSS REFERENCE TO RELATED APPLICATIONS

The present application is a Continuation of PCT/US2021/052378 filed Sep. 28, 2021; which claims the benefit of U.S. Provisional Appln No. 63/084,198 filed Sep. 28, 2020, the disclosures which are incorporated herein by reference their entirety for all purposes.

### FIELD OF THE INVENTION

In general, the present invention provides improved devices, systems, and methods for performing multiple tasks in a workspace accessed via a constrained and/or tortuous access site. In exemplary embodiments, the invention provides nested elongate flexible bodies having articulated portions that can be independently driven via a fluid-drive system. Optionally, the articulated bodies may comprise heart catheters, and one of the catheters may comprise a structural heart therapy catheter. The other catheter may comprise an intracoronary echocardiography (ICE) catheter, and one or both of the catheters may be driven through small-diameter multi-lumen extrusions, with one of the catheters including an asymmetric shaft having one or more relatively small eccentric passage to facilitate fluid communication between multiple independent fluid pressures of a proximal inflation fluid supply system and a distal articulation balloon array via the multi-lumen extrusion.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging, in-part due to the difficulty in accessing (and aligning with) a target tissue using an instrument traversing tortuous vasculature. Alternative minimally invasive surgical technologies include robotic surgery, and robotic systems for manipulation of flexible catheter bodies from outside the patient have also previously been proposed. Some of those prior robotic catheter systems have met with challenges, possibly because of the difficulties in effectively integrating large and complex robotic pull-wire catheter systems into the practice of interventional cardiology as it is currently performed in clinical catheter labs. While the potential improvements to surgical accuracy make these efforts alluring, the capital equipment costs and overall burden to the healthcare system of these large, specialized systems is also a concern. Examples of prior robotic disadvantages that would be beneficial to avoid may include longer setup and overall procedure times, deleterious changes in operative modality (such as a decrease in effective tactile feedback when initially accessing or advancing tools toward an internal treatment site), and the like.

A new technology for controlling the shape of catheters has recently been proposed which may present significant advantages over pull-wires and other known catheter articulation systems. As more fully explained in U.S. Pat. No. 10,646,696, entitled “Articulation Systems, Devices, and Methods for Catheters and Other Uses,” (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister) toward a subset of articulation balloons disposed along segment(s) of the catheter inside the patient so as to induce a desired change in shape. These new technologies may provide catheter control beyond what was previously available, often without having to resort to a complex robotic gantry, without having to rely on pull-wires, and even without having the expense of electric motors. Hence, these new fluid-driven catheter systems appear to provide significant advantages.

Along with the advantages of fluid-driven technologies, significant work is now underway on improved imaging for use by interventional and other doctors in guiding the movement of articulated therapy delivery systems within a patient. Optical, ultrasound, and fluoroscopy systems often acquire planar images (in some cases acquiring multiple planar images on different planes at angularly offset orientations). New three-dimensional (3D) imaging technologies are also now in use. Some of these new imaging systems included 3D image capture devices which acquire images of internal therapy sites from appropriate positions and orientations within the body, and 3D display technologies have been (and are still being) developed and used to show these 3D images.

Despite the advantages of the newly proposed fluid-driven robotic catheter and imaging systems, as with all successes, still further improvements and alternatives would be desirable. In general, it would be beneficial to provide further improved medical devices, systems, and methods, as well as to provide alternative devices, systems, and methods for users to direct the movements of structural heart and other image-guided interventional systems. For example, existing 3D ultrasound systems used for structural heart therapies (such as known transesophageal echocardiography or TEE) often require a separate dedicated imaging specialist to manipulate the probe and control the imaging. Intracoronary echocardiography (ICE) probes can sometimes be advanced into the heart along the vascular pathways by the interventionalist performing a therapy, but commercially available mechanical ICE probe systems can be challenging to steer. These known ICE systems may also rely on a dedicated access site into the vascular system (such as one that is separate from the access site of a therapy tool that will be used to treat tissue). The elongate shaft of such an ICE system may extend parallel next to that of one or more therapeutic interventional tools, including through delicate tissues of the intra-atrial septum. This use of multiple access sites and multiple laterally offset imaging and/or treatment shafts can increase tissue trauma. Hence, technologies which facilitate precise image-guided movements of interventional tools with less trauma would be particularly beneficial. Improved imaging systems that provided some or all of the benefits of existing 2D and 3D imaging with improved efficiencies, ease of use, and accuracy would also be beneficial.

### BRIEF SUMMARY OF THE INVENTION

The present invention generally provides improved devices, systems, and methods for using, training for the use of, planning for the use of, and/or simulating the use of elongate bodies and other tools such as catheters, borescopes, continuum robotic manipulators, rigid endoscopic robotic manipulators, and the like. The image-guided interventional systems and methods provided herein will often make use of nested articulated catheter systems having multiple elongate structural shaft or sheath portions, with each portion supporting an associated therapy tool or imaging tool so as to define an imaging or therapy catheter, such as (for example) an imaging catheter having an image capture device (such as an ultrasound transducer) supported by an imaging catheter sheath portion, and a structural heart therapy catheter having a structural heart therapy tool (such as a Transcatheter Edge-to-Edge Repair or “TEER” tool, a Left Atrial Appendage Closure or “LAAC” tool, or the like) supported by a structural heart therapy catheter shaft portion. Preferably, a portion of one of the catheters is nested within a portion of another of the catheters, and at least one (and ideally both) of the nested catheters is configured for fluid-driven articulation. Optionally, a first tool of the system comprises an image capture device. This first tool may be supported by a shaft portion comprising a first fluid-driven articulated sheath portion. The articulated portion of the sheath is, in turn, supported by an eccentric sheath body having a working lumen that extends distally to a port adjacent the proximal end of the articulated portion. Drive fluid to articulate the sheath can be transmitted distally along the eccentric sheath via one or more eccentric passages that are smaller than the working lumen, ideally via a multi-lumen extrusion disposed in a passage. An articulated shaft supporting a therapeutic tool can be advanced within the working lumen to a target tissue site within the field of view of the image capture device. The fluid transmission channels of the multi-lumen may take much less cross-sectional area of the sheath than would a mechanical pull-wire system, allowing the nested sheath/shaft system to provide access via a single tissue aperture and inducing less trauma than known catheter systems. The technologies described herein can facilitate precise control over catheter-based therapies, including 3D ultrasound image guided structural heart therapies.

In a first aspect, the invention provides a structural heart therapy method for treating a heart of a patient. The heart has a tissue adjacent a chamber, and the method comprises advancing a steerable sheath assembly distally into the chamber. The sheath assembly includes a first therapeutic or diagnostic tool, a port, an articulated sheath portion between the port and the first tool, and a lumen extending proximally from the port. A second diagnostic or therapeutic tool is supported within the chamber with a steerable shaft extending along the lumen and through the port. The shaft includes an articulated portion proximal of the tool, and the first tool is aligned with the tissue by articulating the articulated sheath portion within the chamber. The second tool is aligned with the tissue by articulating the articulated shaft portion within the chamber.

Any number of independent features may be provided, alone or in combination, to enhance functionality of the methods and structures described herein for different purposes. Optionally, the first tool will comprise an image capture device, typically an ultrasound transducer such as an ICE transducer. The transducer will often provide 3D ultrasound images of the tissue while the transducer is oriented toward the tissue along a first imaging axis, and the transducer will also often be used to provide biplanar imaging of the tissue along a first plane and a second plane. Occasionally during use, the second tool will be disposed along or adjacent the first axis between the transducer and the tissue such that the 3D ultrasound images are obstructed. Advantageously, the transducer can be repositioned by articulating the articulated sheath portion so that the transducer is aligned with the tissue along a second imaging axis such that obstruction of the transducer by the second tool is mitigated. Alternatively (or in addition), when the shaft is disposed along or adjacent the first axis between the transducer and the tissue such that the 3D ultrasound images are obstructed, a pose of the articulated shaft portion can be altered such that obstruction of the transducer by the shaft is mitigated. Regardless, the ability to independently articulate the shaft and sheath within the chamber of the heart using a nested system having a monolithic profile of less than a threshold size (such as less than 30 Fr, often being less than 25 Fr, and ideally being 24 Fr or less, 22 Fr or less, or even 20 Fr or less) can provide significant advantages, particularly where each of the shaft and sheath can be articulated with 2 or more, 3 or more, 4 or more, at least 5, or 6 degrees of freedom. Ideally, one or both of the sheath and shaft may be articulated by selective inflation of an articulation balloon array disposed along the associated articulation portion within the chamber of the heart.

The second tool may comprise any of a wide variety of therapeutic or diagnostic tools configured for a structural heart therapy, an arrhythmia therapy, or the like. For example, second tool may optionally comprise an occlusive device configured for occlusion of a left atrial appendage, a paravalvular leak, or a septal defect; a replacement valve configured for use as a mitral valve or tricuspid valve; or a valve repair device. The chamber may optionally comprise a left atrium, a right atrium, a left ventricle, or a right ventricle of the heart.

Preferably, the articulating of the articulated sheath portion or the articulated shaft portion or both is performed by directing inflation fluid distally to a balloon articulation array. Selectively inflating balloons of the array with the inflation fluid can provide a plurality (such as 2, 3, 4, 5, or 6) of articulation degrees of freedom. The inflation fluid can be transmitted distally along the sheath within a multi-lumen shaft or in lumens of the sheath or shaft itself, with the inflation lumens preferably extending along the axis of the sheath and/or shaft eccentrically and being separated from a working lumen of the articulated structure, the lumen typically also being eccentrically offset from a central axis of the structure.

In another aspect, the invention provides a structural heart therapy system for treating a heart of a patient. The heart has a tissue adjacent a chamber, and the system comprises a first therapeutic or diagnostic tool and a second therapeutic or diagnostic tool. A steerable sheath typically includes a proximal sheath body, a port, an articulated sheath portion extending distally of the port, and a lumen extending proximally from the port. The steerable sheath can be configured to support the first tool distally of the articulated sheath portion so as to facilitate aligning the first tool with the tissue by articulating the articulated sheath portion within the chamber. A steerable shaft can be slidably receivable within the lumen, and the shaft can include a proximal shaft body and be configured to support the second tool with an articulated portion between the second tool and the shaft body so as to facilitate aligning the second tool with the tissue by articulating the articulated shaft portion within the chamber. Preferably, the first diagnostic tool comprises an image capture device and has a profile in a range from 14 Fr to 26 Fr.

In yet another aspect, the invention provides a fluid-driven retrograde catheter articulation method comprising introducing an elongate shaft distally into a patient body. The shaft has a proximal end and a distal end with an axis therebetween, and also has an eccentric fluid passage and a cross-section with an indent so that the shaft defines a laterally open channel. Fluid is transmitted along the eccentric lumen, and along the articulated portion to an actuator system. The articulated portion is driven, using the fluid in the actuator, between a first articulation state and a second articulation state. The articulated portion in the first state extends proximally in the channel so as to have a profile suitable for insertion into a patient body. A proximal end of the articulated portion is, in the second state, laterally offset from the shaft.

Preferably, the elongate shaft includes a plurality of eccentric fluid passages, and the actuator system comprises an articulation balloon array. A structural heart tool can be supported by the proximal end of the articulated portion, and selectively inflating subsets of balloons of the array with the passages can move the tool with a plurality of degrees of freedom. Optionally, an image capture device can be moved by articulating an imaging articulation portion distal of the distal end of the shaft. The tool can be imaged with the image capture device, and the imaging articulation portion can be articulated by an imaging articulation balloon array.

In yet another aspect, the invention provides a fluid-driven retrograde articulated catheter system comprising an elongate shaft having a proximal end and a distal end with an axis therebetween. The shaft can have a cross-section with an indent so as to define a laterally open channel and an eccentric fluid passage. The channel and fluid passage extend along the axis. An articulated distal portion has a proximal end and a distal end with an articulated portion axis extending therebetween. The distal end of the articulated portion is supported by the distal end of the sheath. An actuator system is disposed along the articulated distal portion in fluid communication with the fluid passage so that transmission of fluid along the passage drives the articulated portion between a first articulation state and a second articulation state. The articulated portion in the first state extends proximally in the channel so as to have a profile suitable for insertion into a patient body. The proximal end of the articulated portion in the second state is laterally offset from the shaft.

In another aspect, the invention provides a catheter system for treating a tissue of a patient. The system comprises a first therapeutic or diagnostic tool and a second therapeutic or diagnostic tool. An elongate channel body has a proximal end and a distal end with an axis extending therebetween, and a channel extends proximally from the distal end. The channel body can support a plurality of fluid drive lumens extending axially and offset laterally from the channel. A first elongate articulatable body is removably receivable within the channel, the articulatable body configured to support the first tool with the articulatable body extending between the first tool and the distal end of the channel body so as to move the first tool with lateral articulation of the articulatable body. A second elongate articulatable body extends axially from the distal end of the channel body so as to facilitate aligning the second tool with the tissue by laterally articulating the articulatable body with fluid pressure from the fluid drive lumens.

Optionally, a port is disposed at the distal end of the channel body. The channel body may comprise a proximal sheath body and the channel may comprise a sheath lumen extending proximally from the port. The first articulatable body can comprise a steerable shaft slidably received within the sheath lumen. Such an arrangement can include axially slidable nested sheath and shaft catheters described herein, often to provide the benefit of two independently articulatable catheter structures within an internal therapy site while limiting tissue trauma along a vascular or other access pathway to a single outer tissue-engaging sheath surface.

In some embodiments, the port may be defined by a metal axial offset structure having a proximal tubular body affixed to the proximal sheath body, a distal tubular body affixed to an articulated sheath portion (the articulated sheath portion comprising the second articulatable body), and an eccentric offset structural member separating the proximal sheath body axially from the articulated sheath portion. The eccentric offset structural member may support the fluid drive lumens in fluid communication with articulation balloons of the articulated sheath portion so as to allow fluid from the fluid drive lumens to laterally bend the articulated sheath portion and align the second tool with the tissue.

Alternatively, the channel may comprise a laterally open channel, and the second articulatable body may extend proximally within the laterally open channel so that the second tool at the proximal end of the second articulatable body is driven laterally from the laterally open channel and into alignment with the tissue when the drive fluid is direct to articulation balloons of the second articulatable body. Such systems may take advantage of any of the features of systems having multiple independently articulatable catheter sheaths and/or shafts described herein, particularly those that take advantage of a catheter body (sheath or shaft) that extends in a proximal or retrograde orientation from the distal end of a support structure having a laterally open channel.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIG. 2 is a simplified schematic illustration of components of a helical balloon assembly, showing how an extruded multi-lumen shaft can provide fluid to laterally aligned subsets of balloons within an articulation balloon array of a catheter.

FIGS. 3A-3C schematically illustrate helical balloon assemblies supported by flat springs and embedded in an elastomeric polymer matrix, and also show how selective inflation of subsets of the balloons can elongate and laterally articulate the assemblies.

FIG. 4 is a perspective view of a robotic catheter system in which a catheter is removably mounted on a driver assembly, and in which the driver assembly includes a driver encased in a sterile housing and supported by a stand.

FIG. 5 schematically illustrates a robotic catheter system and transmission of signals between the components thereof so that input from a user induces a desired articulation.

FIG. 6 and FIG. 6A schematically illustrates a nested robotic catheter system and method including a fluid-driven articulated structural heart therapy catheter which passes through a working lumen of a fluid-driven articulated ultrasound imaging catheter.

FIG. 7 schematically illustrates a nested robotic catheter system and method in which a mechanical pull-wire structural heart therapy catheter passes through a working lumen of a fluid-driven articulated ultrasound imaging catheter.

FIG. 8 and FIG. 8A schematically illustrates a retrograde robotic catheter system and method in which an articulated catheter portion extends proximally in a channel of a shaft having a C-shaped cross-section for insertion into the patient, with eccentric fluid passages extending within the C-shaped shaft being used to articulate the articulated portion to position a structural heart tool (or other tool) in a chamber of the heart (or other worksite).

FIG. 9 schematically illustrates a catheter system having a retrograde tool articulated portion which can be removably received in a C-shaped channel as seen in FIG. 8, along with a distal imaging portion as seen in FIG. 6.

FIGS. 10A-10C show a side view, a perspective view, and a cross-sectional view of an alternative multi-catheter system, respectively, in which a port in an articulatable sheath is defined by an axial offset structural member extending between a non-articulated proximal portion of the sheath and an articulated distal portion of the sheath.

FIGS. 10D-10F show a solid perspective view, a transparent perspective view, and an end view of the exemplary axial offset.

### DETAILED DESCRIPTION OF THE INVENTION

The improved devices, systems, and methods for controlling, image guidance of, inputting commands into, and simulating movement of powered and robotic devices will find a wide variety of uses. The elongate tool-supporting structures described herein will often be flexible, typically comprising catheters suitable for insertion in a patient body. Exemplary systems will be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber).

Alternative applications may include use in steerable supports of image acquisition devices such as for trans-esophageal echocardiography (TEE), intra-coronary echocardiography (ICE), and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The robotic systems described herein will often include an input device, a driver, and an articulated catheter or other robotic manipulator supporting a diagnostic or therapeutic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool-supporting catheter along a plurality of fluid channels. Optionally, the drive signals may comprise electromagnetic, optical, or other signals, preferably (although not necessarily) in combination with fluidic drive signals. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system will be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

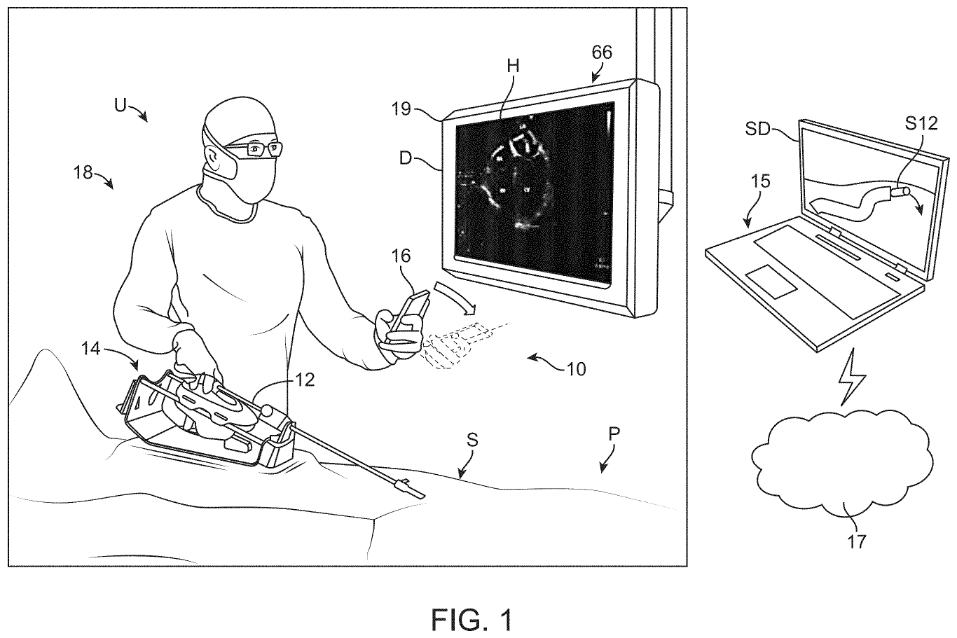

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by sliding the catheter relative to a stand of the driver assembly, while viewing a distal end of the catheter and the surrounding tissue in a display D. As will be described below, user U may alternatively manually rotate the catheter body about its axis in some embodiments.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being transmitted along the catheter body as a hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MRI), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet that can be supported by or near driver assembly 14, incorporating the input device, computer, and one or both of displays D, SD into a workstation near the patient, shielded from the imaging system, and/or remote from the patient, or the like.

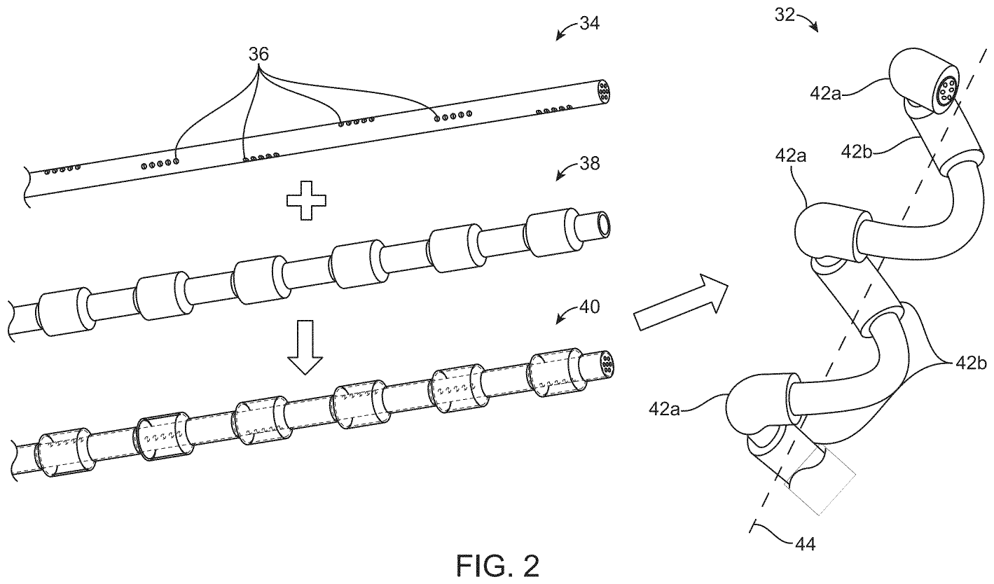

Referring now to FIG. 2, the components of, and fabrication method for production of, an exemplary balloon array assembly (sometimes referred to herein as a balloon string 32) can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 34 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using a compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. Each lumen of the shaft may be associated with between 3 and 50 balloons, typically from about 5 to about 30 balloons. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

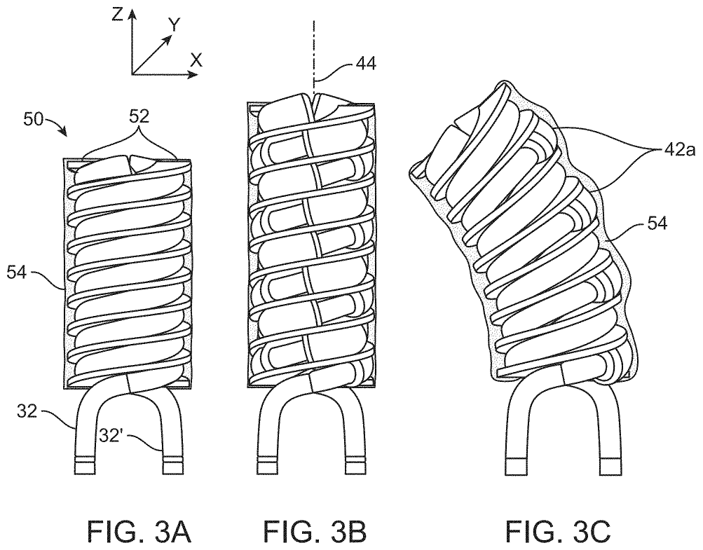

Referring now to FIGS. 3A, 3B, and 3C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 3A and 3B, inflation of subsets of the balloons surrounding the axis of segment 50 can induce axial elongation of the segment. As can be understood with reference to FIGS. 3A and 3C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 or 7 lumens), so that the total balloon array may include a series of independently articulatable segments (each having 3 or 4 dedicated lumens of one of the multi-lumen shafts, for example). Optionally, from 2 to 4 modular, axially sequential segments may each have an associated tri-lumen shaft, with the tri-lumen shaft extending axially in a loose helical coil through the lumen of any proximal segments to accommodate bending and elongation. The segments may each include a single helical balloon string/multilumen shaft assembly (rather than having a dual-helix configuration). Multi-lumen shafts for driving of distal segments may alternatively wind proximally around an outer surface of a proximal segment, or may be wound parallel and next to the multi-lumen shaft/balloon tube assemblies of the balloon array of the proximal segment(s).

Referring still to FIGS. 3A, 3B, and 3C, articulated segment 50 optionally includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Alternatively (or in addition to a relatively soft matrix), a thin layer of relatively high-strength elastomer can be applied to the assembly (prior to, after, or instead of the soft matrix), optionally while the balloons are in an at least partially inflated state. Advantageously, matrix 54 can help maintain overall alignment of the balloon array and springs within the segment despite segment articulation and bending of the segment by environmental forces. Regardless of whether or not a matrix is included, an inner sheath may extend along the inner surface of the helical assembly, and an outer sheath may extend along an outer surface of the assembly, with the inner and/or outer sheaths optionally comprising a polymer reinforced with wire or a high-strength fiber in a coiled, braid, or other circumferential configuration to provide hoop strength while accommodating lateral bending (and preferably axial elongation as well). The inner and outer sheaths may be sealed together distal of the balloon assembly, forming an annular chamber with the balloon array disposed therein. A passage may extend proximally from the annular space around the balloons to the proximal end of the catheter to safely vent any escaping inflation media, or a vacuum may be drawn in the annular space and monitored electronically with a pressure sensor to inhibit inflation flow if the vacuum deteriorates.

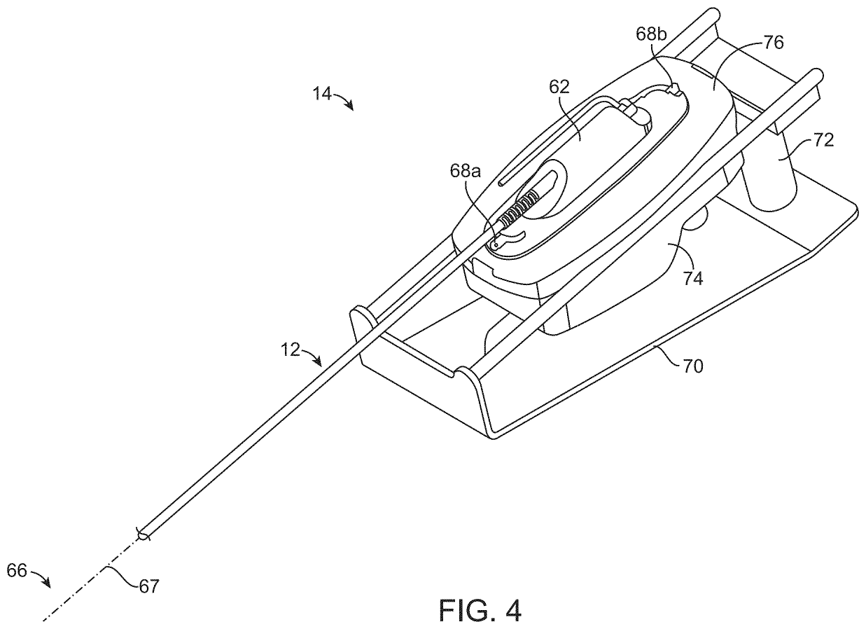

Referring now to FIG. 4, a proximal housing 62 of catheter 12 and the primary components of driver assembly 14 can be seen in more detail. Catheter 12 generally includes a catheter body 64 that extends from proximal housing 62 to an articulated distal portion 66 (see FIG. 1) along an axis 67, with the articulated distal portion preferably comprising a balloon array and the associated structures described above. Proximal housing 62 also contains first and second rotating latch receptacles 68a, 68b which allow a quick-disconnect removal and replacement of the catheter. A variety of alternative latching arrangements may also be provided. The components of driver assembly 14 visible in FIG. 4 include a sterile housing 70 and a stand 72, with the stand supporting the sterile housing so that the sterile housing (and components of the driver assembly therein, including the driver) and catheter 12 can move axially along axis 67. Sterile housing 70 generally includes a lower sterile housing 74 and a sterile junction having a sterile barrier 76. Sterile junction 76 releasably latches to lower sterile housing 74 and includes a sterile barrier body that extends between catheter 12 and the driver contained within the sterile housing. Along with components that allow articulation fluid flow to pass through the sterile fluidic junction, the sterile barrier may also include one or more electrical connectors or contacts to facilitate data and/or electrical power transmission between the catheter and driver, such as for articulation feedback sensing, manual articulations sensing, or the like. The sterile housing 74 and sterile junction 76 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 72 will often comprise a metal, such as a stainless steel, aluminum, or the like for repeated sterilizing and use.

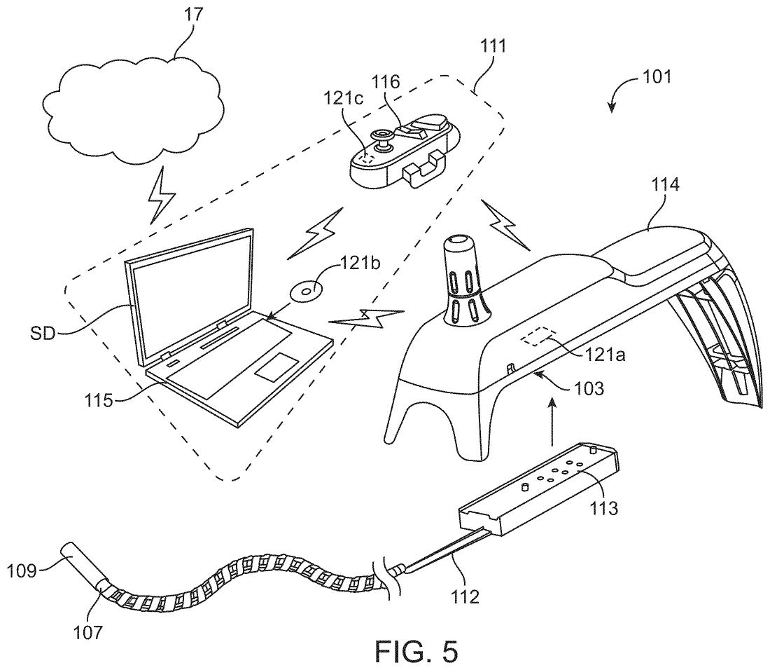

Referring now to FIG. 5, components of a simulation system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114.

Alternative catheter 112 can be replaceably coupled with alternative driver assembly 114. When simulation system 101 is used for driving an actual catheter, the coupling may be performed using a quick-release engagement between an interface 113 on a proximal housing of the catheter and a catheter receptacle 103 of the driver assembly. An elongate body of catheter 112 has a proximal/distal axis as described above and a distal receptacle 107 that is configured to support a therapeutic or diagnostic tool 109 such as a structural heart tool for repairing or replacing a valve of a heart. The tool receptacle may comprise an axial lumen for receiving the tool within or through the catheter body, a surface of the body to which the tool is permanently affixed, or the like. Alternative drive assembly 114 may be wireless coupled to a simulation computer 115 and/or a simulation input device 116, or cables may be used for transmission of data.

When alternative catheter 112 and alternative drive system 114 comprise virtual structures, they may be embodied as modules of software, firmware, and/or hardware. The modules may optionally be configured for performing articulation calculations modeling performance of some or all of the actual clinical components as described below, and/or may be embodied as a series of look-up tables to allow computer 115 to generate a display effectively representing the performance. The modules will optionally be embodied at least in-part in a non-volatile memory of a simulation-supporting alternative drive assembly 121a, but some or all of the simulation modules will preferably be embodied as software in non-volatile memories 121b, 121c of simulation computer 115 and/or simulation input device 116, respectively. Coupling of alternative virtual catheters and tools can be performed using menu options or the like. In some embodiments, selection of a virtual catheter may be facilitated by a signal generated in response to mounting of an actual catheter to an actual driver.

Simulation computer 115 preferably comprises an off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other simulation computers 115 and/or one or more clinical drive assemblies 14. Simulation computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Simulation input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3^{rd} degree of freedom), a tablet or phone having an X-Y touch screen (optionally with AR capabilities such as being compliant with ARCor from Google, ARKit from Apple, or the like to facilitate input of translation and/or rotation, along with multi-finger gestures such as pinching, rotation, and the like), a gamepad, a 3D mouse, a 3D stylus, or the like. Proprietary code may be loaded on the simulation input device (particularly when a phone, tablet, or other device having a touchscreen is used), with such input device code presenting menu options for inputting additional commands and changing modes of operation of the simulation or clinical system. A simulation input/output system 111 may be defined by the simulation input device 116 and the simulation display SD.

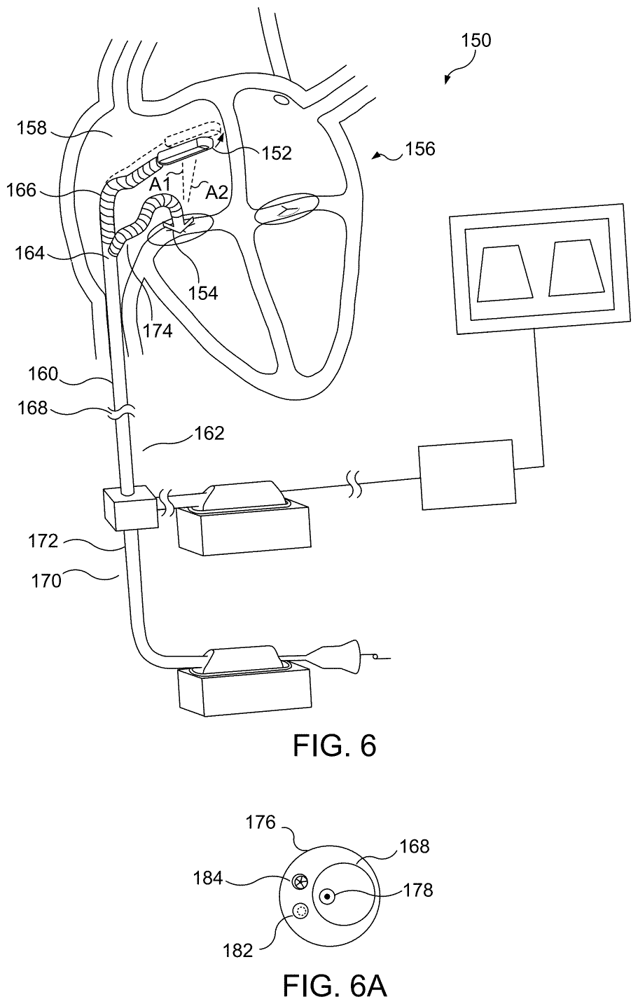

Referring now to FIG. 6, a nested ultrasound/structural heart therapy delivery system 150 makes use of many of the components described above to provide a method for both imaging and image-guided therapy delivery. More generally, nested catheter systems can be used to position and orient a first therapeutic or diagnostic tool relative (such as an ultrasound transducer 152) relative to a second therapeutic or diagnostic tool (such as an edge-to-edge valve leaflet clip 154 or other structural heart therapy tool). During use, the nested catheters support and move transducer 152 and clip 154 in a chamber of heart 156, such as in right atrium 158. This can facilitate, for example repositioning of the ultrasound transducer 152 from an initial position in which the clip is viewed along an initial viewing axis A1 (in which the clip blocks a portion of the desired view of the target valve tissue) to an improved viewing axis A2 (in which the offset field of view provides a better view of the clip and/or target tissue).

Referring now to FIGS. 6 and 6A, nested catheter system 150 includes a steerable sheath 160 having a proximal sheath body 162, a port 164, an articulated sheath portion 166 extending distally of the port, and a lumen 168 extending proximally from the port. Steerable sheath 160 is configured to support transducer 152 distally of the articulated sheath portion so as to facilitate aligning the transducer with the target tissue by articulating the articulated sheath portion within the right atrium. Nested catheter system 150 also includes a steerable shaft 170 slidably receivable within lumen 168 of the imaging sheath, the shaft including a proximal shaft body 172 and configured to support the clip 154 with an articulated portion 174 between the clip and the shaft body so as to facilitate aligning the clip with the target tissue by articulating the articulated shaft portion within the atrium. A cross-section 176 of sheath body portion 162 shows how lumen 168 is eccentric relative to an axis 178 and an outer profile of the sheath, along with an eccentric passage 182 containing the multi-lumen fluid supply for an articulation balloon of the imaging articulation portion, and one or more additional eccentric passages 184 for imaging wires to transmit the images from transducer 152 or the like.

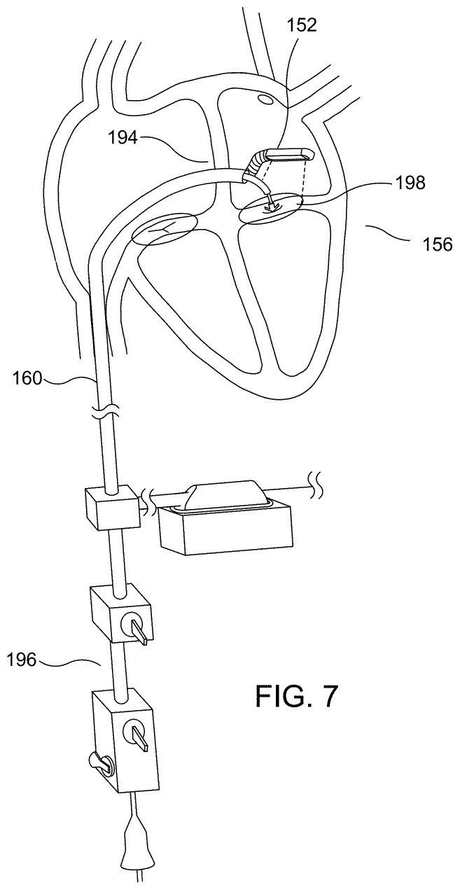

Referring now to FIG. 7, an alternative nested imaging/therapy catheter system 190 includes an imaging catheter 160 similar to that described above, but here configured for use in a left atrium 192 of heart 156 via a femoral/trans-septal access route 194. Rather than a fluid-articulated therapy catheter, a mechanical pull-wire structural heart therapy catheter 196 is provided which relies on rotation of knobs, rotations of housings, sliding of housings, and the like proximal of the patient body to position and orient a tool 198 relative to target tissue and in a field of view of transducer 152. Suitable mechanical structural heart therapy catheters may include those commercially available from Abbott, Edwards Life Sciences, and others.

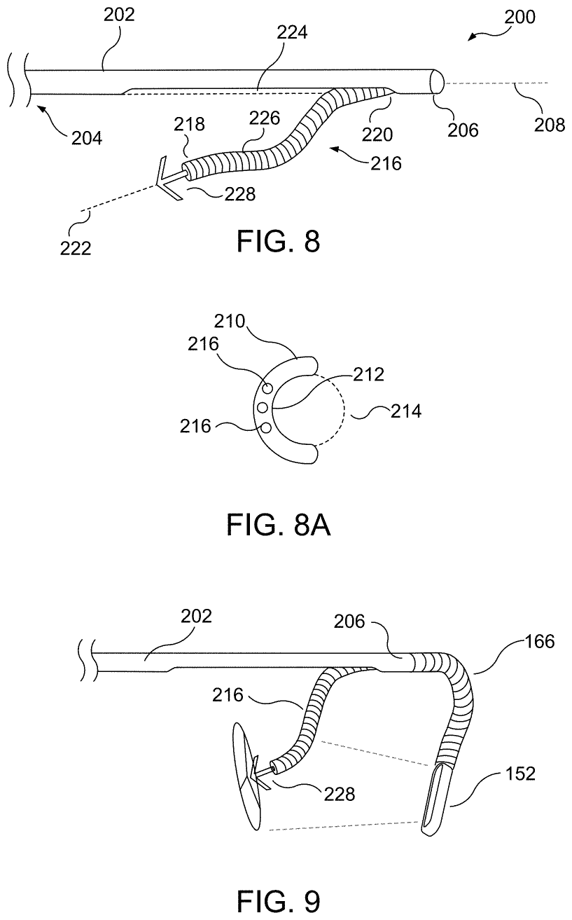

Referring now to FIGS. 8 and 8A, a fluid-driven retrograde articulated catheter 200 system includes an elongate shaft 202 having a proximal end 204 and a distal end 206 with an axis 208 therebetween. The shaft has one or more eccentric fluid passage 216 and a cross-section 210 with an indent 212 so as to define a laterally open channel 214. The channel and fluid passage extend along the axis. Proximal catheter shaft 202 will often be at least somewhat laterally flexible, but will often not be actively articulated. An articulated distal portion 226 may have a proximal end 218 and a distal end 220 with an articulated portion axis 222 extending therebetween. The distal end 220 of the articulated portion 216 may be supported by the distal end 206 of the sheath 202. An actuator system can be disposed along the articulated distal portion in fluid communication with the fluid passage so that transmission of fluid along the passage drives the articulated portion as described above. Articulated portion 226 can be configured to be driven between a first articulation state 224 and a second articulation state, the articulated portion in the first state extending proximally in the channel 214 so as to have a profile suitable for insertion into a patient body. The proximal end 218 of the articulated portion 216 in the second state can be laterally offset from the shaft 202.

As shown in FIG. 8, the proximal end 218 of the articulated portion 226 can support a structural heart tool such as a clip 228. As can be understood with reference to the descriptions of articulation systems above, the elongate shaft 202 typically includes a plurality of eccentric fluid passages defined by a multi-lumen shaft or the like, and the actuator system will often include an articulation balloon array, with subsets of balloons of the array being in fluid communication with the passages of the multi-lumen shaft so as to facilitate positioning and orienting of the tool 228 with a plurality of degrees of freedom.

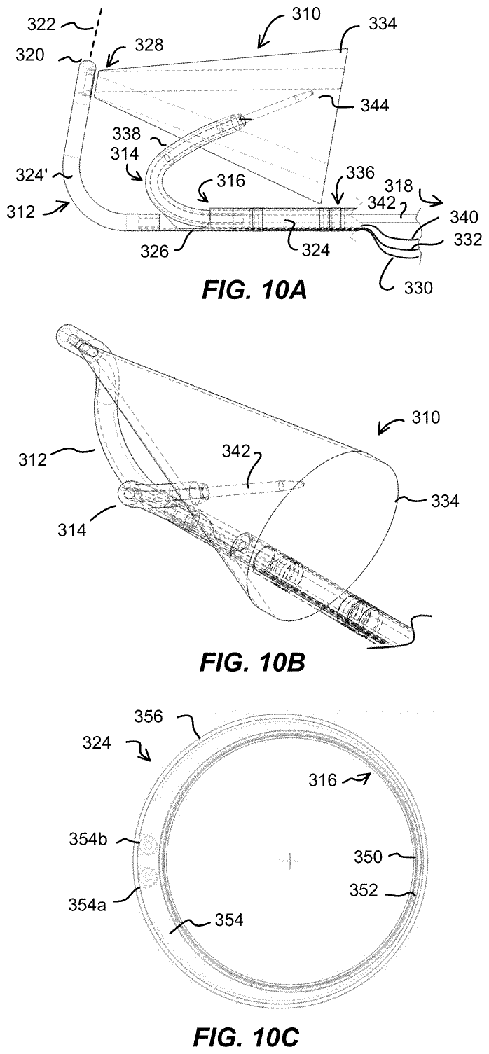

As shown in FIG. 9, imaging articulating catheter components of the nested catheter system of FIG. 6 can be combined with the retrograde catheter system of FIG. 8 to provide an advantageous monolithic imaging/retrograde catheter system. More specifically, an image capture device 152 can be supported by an imaging articulation portion 166 distal of the distal end 206 of the shaft 202. As described above, an imaging articulation balloon array can be disposed along the imaging articulation portion 166. The image capture device 152 can be configured to image the tool 228 with the imaging articulation array driving the imaging articulation portion 216 into alignment with the tool. Drive fluid for imaging articulation portion 166 can be transmitted along the shaft 202 in another multi-lumen included in a dedicated passage 216, and both multi-lumens may be supplied with inflation fluid from a single driver having sufficient fluid channels, or a separate driver may be coupled for each of the articulated catheter portions.

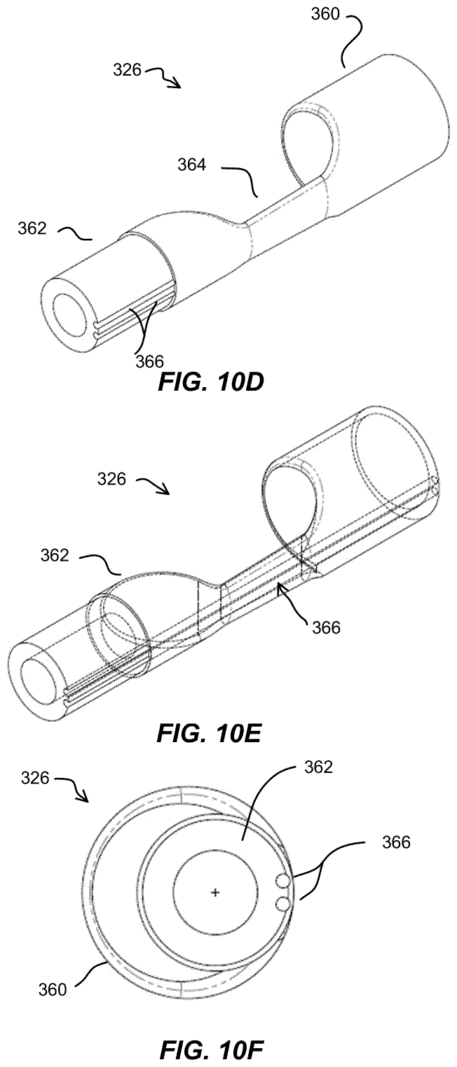

Referring now to FIGS. 10A, 10B, and 10C, an exemplary imaging and therapy catheter system 310 includes an articulatable imaging catheter 312 and an articulatable therapy catheter 314 slidably nested within a sheath lumen 316 of imaging catheter 312.

Imaging catheter 312 (like most or all of the other catheters and elongate catheter portions and described herein) has a proximal end 318 and a distal end 320 with an axis 322 extending therebetween. An elongate non-articulated proximal sheath portion 324 of imaging catheter 312 defines an axial channel in the form of sheath lumen 316, which removably receives the therapy catheter 314 therein so that the therapy catheter can slide axially and rotate about its axis. An elongate distal articulatable sheath portion 324′ of imaging catheter 312 extends distally of the proximal sheath portion 324 with an offset structural member 326 extending axially therebetween. Articulatable sheath portion 324′ supports a tool, preferably an image capture device, and ideally an ultrasound transducer 328 near the distal end of the imaging catheter so as to facilitate positioning of the ultrasound transducer in alignment with tissues targeted for imaging and/or treatment, often by laterally bending the distal articulatable sheath portion in response to fluid pressure in a series of fluid drive channels using articulation balloons as described herein, with the fluid drive channels typically being included in a multi-lumen extrusion 332. Ultrasound transducer 328 may comprise an echocardiography transducer, and imaging catheter 312 may comprise an intra-coronary echocardiography (ICE) catheter, with the transducer ideally transmitting real-time three-dimensional (RT3D, sometimes called 4D) image signals and/or bi-plane image signals toward a display along an imaging cable 330. The image signals define 3D and/or 2D images within a field of view 334.

Therapy catheter 314 includes a non-articulated proximal shaft portion 336 and a distal articulated shaft portion 338, with the distal shaft portion again preferably being laterally articulatable by varying pressures in an array of articulation balloons within the distal shaft portion. The shaft articulation pressures may be transmitted to the distal shaft portion along the lumens of a multi-lumen extrusion 340 that extends axially within the proximal shaft portion. An elongate flexible tool shaft 342 may optionally extend axially within a lumen of therapy catheter 314, so that a therapeutic tool 344 at the distal end of the tool shaft can be moved by lateral bending of the articulatable shaft portion into alignment with a tissue within the field of view 334 of the imaging catheter.

An eccentric cross-section of exemplary proximal sheath portion 324 of imaging catheter 312 can be understood with reference to FIG. 10C. A multi-layer catheter build here provides desired torsional stiffness and axial stiffness for pushability and torquability. The build also allows the guide to be pre-set with a desired lateral bend suitable for trans-septal crossing (not shown) and imaging in the left atrium, with the bend being resilient to allow straightening of the bend for insertion. The exemplary layers include a low-friction polymer inner liner 350 comprising polytetrafluoroethylene (PTFE) or the like bordering lumen 316. A braid-reinforced polymer layer 352 over the inner liner 350 comprises a stainless, non-magnetic alloy such as Elgiloy™, or other metal braid and a thermoplastic or thermoset polymer such as a nylon, a urethane, a Pebax™, a Pelathane™, or other polymer matrix thermally reflowed over the inner liner. An eccentric polymer catheter body layer 354 over the braid-reinforced polymer layer 352 comprises a resilient thermoplastic or thermoset polymer such as a Pebax™, a Pelathane™, or the like. Eccentric body later 354 has an inner diameter that is laterally offset from its outer diameter and defines one or more (preferably two, three, or more) channels 354a, 354b that can receive a multi-lumen fluid drive extrusion, an ultrasound transducer cable, a guidewire, an articulation wire, or a combination of these (and optionally other) structures. An outer polymer layer 356 may provide a desirable outer surface with limited stiction during insertion and torsion of the imaging catheter, with the outer surface optionally comprising a PTFE layer or a thin polymer layer having friction-reducing compounding materials therein. Radio-opaque and/or echogenic markers may be disposed over or under outer layer 356.

Referring now to FIGS. 10D-10F, exemplary offset member 326 includes a proximal body 360 which optionally comprises a tubular body configured to be affixed to the distal end of the non-articulated proximal sheath portion. A distal body 362 optionally comprises a tubular body configured to be affixed to the proximal end of the articulatable sheath portion. An axial offset intermediate body 364 extends between the proximal body 360 and the distal body 362 so as to support the articulatable sheath portion, and includes one or more axial channels 366 that define or contain the plurality of axial fluid drive lumens, typically in the form of a polymer multi-lumen extrusion, and the transducer cables or other signal and/or motion transmission structures. Offset member 326 may optionally comprise a metal such as a stainless steel, a non-magnetic alloy such as an Elgiloy™, a polymer such as a nylon, a polyetheretherketone or PEEK, or the like. One or both of the tubular bodies may have eccentric inner and outer diameters, and the bodies may be of different outer profiles, for example, when the articulatable sheath portion has a smaller profile than a profile of the proximal sheath portion. Note that intermediate body 364 may comprise a laterally open channel to enhance stiffness of a polymer structure, and/or if the articulatable portion of the sheath extends proximally in a retrograde configuration similar to that shown above.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. A structural heart therapy method for treating a heart of a patient, the heart having a tissue adjacent a chamber, the method comprising:
advancing a steerable sheath assembly distally into the chamber, the sheath assembly including a first therapeutic or diagnostic tool, a port, an articulated sheath portion between the port and the first tool, and a lumen extending proximally from the port;
supporting a second diagnostic or therapeutic tool within the chamber with a steerable shaft extending along the lumen and through the port, the shaft including an articulated portion proximal of the tool;
aligning the first tool with the tissue by articulating the articulated sheath portion within the chamber; and
aligning the second tool with the tissue articulating the articulated shaft portion within the chamber.

2. The method of claim 1, wherein the first tool comprises an image capture device.

3. The method of claim 2, wherein the image capture device comprises an ultrasound transducer, and further comprising obtaining 3D ultrasound images of the tissue with the transducer while the transducer is oriented toward the tissue along a first imaging axis.

4. The method of claim 3, wherein the second tool is disposed along or adjacent the first orientation axis between the transducer and the tissue such that the 3D ultrasound images are obstructed, and further comprising repositioning the transducer by articulating the articulated sheath portion so as that the transducer is aligned with the tissue along a second imaging axis such that obstruction of the transducer by the second tool is mitigated.

5. The method of claim 3, wherein the shaft is disposed along or adjacent the first orientation axis between the transducer and the tissue such that the 3D ultrasound images are obstructed, and further comprising altering a pose of the articulated shaft portion such that obstruction of the transducer by the shaft is mitigated.

6. The method of claim 1, wherein the second tool comprises an occlusive device configured for occlusion of a left atrial appendage, a paravalvular leak, or a septal defect; a replacement valve configured for use as a mitral valve or tricuspid valve; or a valve repair device.

7. The method of claim 1, wherein the chamber comprises a left atrium or a right atrium of the heart.

8. The method of claim 1, wherein the articulating of the articulated sheath portion or the articulated shaft portion or both is performed by directing inflation fluid distally to a balloon articulation array, and further comprising selectively inflating balloons of the array with the inflation fluid to provide a plurality of articulation balloon degrees of freedom.

9. The method of claim 8, wherein the inflation fluid is transmitted distally along the sheath within a multi-lumen shaft, wherein the multilumen shaft is eccentrically separated from the lumen and the lumen is eccentrically offset from a central axis of the sheath.

10. A structural heart therapy system for treating a heart of a patient, the heart having a tissue adjacent a chamber, the system comprising:
a first therapeutic or diagnostic tool and a second therapeutic or diagnostic tool;
a steerable sheath including a proximal sheath body, a port, an articulated sheath portion extending distally of the port, and a lumen extending proximally from the port, the steerable sheath configured to support the first tool distally of the articulated sheath portion so as to facilitate aligning the first tool with the tissue by articulating the articulated sheath portion within the chamber; and
a steerable shaft slidably receivable within the lumen, the shaft including a proximal shaft body and configured to support the second tool with an articulated portion between the second tool and the shaft body so as to facilitate aligning the second tool with the tissue by articulating the articulated shaft portion within the chamber.

11. The system of claim 10, wherein the first diagnostic tool comprises an image capture device and has a profile in a range from 14 Fr to 26 Fr.

12. A fluid-driven retrograde catheter articulation method comprising:
introducing an elongate shaft distally into a patient body, the shaft having a proximal end and a distal end with an axis therebetween, shaft also having an eccentric fluid passage and a cross-section with an indent so that the shaft defines a laterally open channel;
transmitting fluid along the axis of the eccentric lumen, and along the articulated portion to an actuator system; and
driving, using the fluid in the actuator, the articulated portion between a first articulation state and a second articulation state, the articulated portion in the first state extending proximally in the channel so as to have a profile suitable for insertion into a patient body, a proximal end of the articulated portion in the second state being laterally offset from the shaft.

13. The method of claim 12, wherein the elongate shaft includes a plurality of eccentric fluid passages, and wherein the actuator system comprises an articulation balloon array, and further comprising supporting a structural heart tool with the proximal end of the articulated portion, and selectively inflating subsets of balloons of the array with the passages so as to move the tool with a plurality of degrees of freedom.

14. The method of claim 13, further comprising aligning an image capture device by articulating an imaging articulation portion distal of the distal end of the shaft, and imaging the tool with the image capture device, wherein the imaging articulation portion is articulated by an imaging articulation balloon array.

15. A fluid-driven retrograde articulated catheter system comprising:
an elongate shaft having a proximal end and a distal end with an axis therebetween, the shaft having a cross-section with an indent so as to define a laterally open channel and an eccentric fluid passage, the channel and fluid passage extending along the axis;
an articulated distal portion having a proximal end and a distal end with an articulated portion axis extending therebetween, the distal end of the articulated portion supported by the distal end of the sheath; and
an actuator system disposed along the articulated distal portion in fluid communication with the fluid passage so that transmission of fluid along the passage drives the articulated portion between a first articulation state and a second articulation state, the articulated portion in the first state extending proximally in the channel so as to have a profile suitable for insertion into a patient body, the proximal end of the articulated portion in the second state being laterally offset from the shaft.

16. The system of claim 15, wherein the proximal end of the articulated portion supports a structural heart tool, and wherein the elongate shaft includes a plurality of eccentric fluid passages, and wherein the actuator system comprises an articulation balloon array, subsets of balloons of the array being in fluid communication with the passages so as to facilitate positioning and orienting of the tool with a plurality of degrees of freedom.

17. The system of claim 16, further comprising an image capture device supported by an imaging articulation portion distal of the distal end of the shaft, an imaging articulation balloon array disposed along the imaging articulation portion, the image capture device configured to image the tool with the imaging articulation array driving the imaging articulation portion into alignment with the tool.

18. A catheter system for treating a tissue of a patient, the system comprising:
a first therapeutic or diagnostic tool and a second therapeutic or diagnostic tool;
an elongate channel body having a proximal end and a distal end with an axis extending therebetween, wherein a channel extends proximally from the distal end, the channel body supporting a plurality of fluid drive lumens extending axially and offset laterally from the channel;
a first elongate articulatable body removably receivable within the channel, the articulatable body configured to support the first tool with the articulatable body extending between the first tool and the distal end of the channel body to move the first tool with lateral articulation of the articulatable body; and
a second elongate articulatable body extending axially from the distal end of the channel body so as to facilitate aligning the second tool with the tissue by laterally articulating the articulatable body with fluid pressure from the fluid drive lumens.

19. The catheter system of claim 18, wherein a port is disposed at the distal end of the channel body, wherein the channel body comprises a proximal sheath body and the channel comprises a sheath lumen extending proximally from the port, and wherein the first articulatable body comprises a steerable shaft slidably received within the sheath lumen.

20. The catheter system of claim 19, wherein the port is defined by a metal axial offset structure having a proximal tubular body affixed to the proximal sheath body, a distal tubular body affixed to an articulated sheath portion, the articulated sheath portion comprising the second articulatable body, and an eccentric offset structural member separating the proximal sheath body axially from the articulated sheath portion, the eccentric offset structural member supporting the fluid drive lumens in fluid communication with articulation balloons of the articulated sheath portion so as to laterally bend the articulated sheath portion and align the second tool with the tissue.

21. The nested catheter of claim 18, wherein the channel comprises a laterally open channel, and wherein the second articulatable body extends proximally within the laterally open channel so that the second tool at the proximal end of the second articulatable body is driven laterally from the laterally open channel and into alignment with the tissue when the drive fluid is direct to articulation balloons of the second articulatable body.
